(19) United States
(12) Patent Application Publication
(10) Pub. No.: US 2022/0184170 A1
(43) Pub. Date: Jun. 16, 2022

(54) FORMULATION TO ENHANCE THE BIOAVAILIBITY AND STABILITY OF CURCUMINOIDS AND/OR ITS DERIVATIVES THEREOF

(71) Applicant: NaishadhKumar Ramubhai Patel, Ahmedabad (IN)

(72) Inventor: NaishadhKumar Ramubhai Patel, Ahmedabad (IN)

(21) Appl. No.: 17/437,415

(22) PCT Filed: Mar. 21, 2020
(86) PCT No.: PCT/IN2020/050268

§ 371 Date: Sep. 8, 2021

(30) Foreign Application Priority Data
Mar. 24, 2019 (IN) 201821048953

(51) Int. Cl. A61K 36/9066; A61K 31/12; A61K 47/44; A61K 9/00; A61K 9/48

(52) U.S. Cl. CPC A61K 36/9066; A61K 31/12; A61K 9/4875; A61K 9/0053; A61K 47/44

(57) ABSTRACT

The present invention provides a formulation comprising turmeric extract, which exhibits enhanced aqueous solubility and bioavailability, which is useful for therapeutic applications and overcomes limitations of the prior art. The formulation further comprises at least one of piperine, turmeric oil, acrysol K-140, and bees wax.

### FIELD OF THE INVENTION

The present invention relates to formulations comprising turmeric extract. Particularly, the present invention relates to formulations comprising turmeric extract exhibiting enhanced aqueous solubility, bioavailability, and stability.

### BACKGROUND OF THE INVENTION

Curcuminoids, also referred to as diferuloylmethanes, and are bis-α, β unsaturated β-diketone compounds derived from the turmeric (Curcuma longa), an anti-oxidant-rich herb that has been widely used in India as a spice for thousands of years. The partially purified natural complex of diaryl heptanoid derivatives isolated from Turmeric, contains NLT 95.0% of curcuminoids, calculated on dried basis, as the sum of curcumin, desmethoxycurcumin, and bisdesmethoxycurcumin. It contains NLT 70.0% and NMT 80.0% of curcumin, NLT 15.0% and NMT 25% of desmethoxycurcumin, and NLT 2.5% and NMT 6.5% of bisdesmethoxycurcumin.

Curcumin is a yellow color polyphenolic compound having chemical formula, 1,7-bis(4-hydroxy-3-methoxyphenyl)-1,6-heptadiene-3,5-dione having structure:

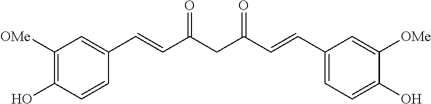 [SMILES: COc1cc(/C=C/C(=O)CC(=O)/C=C/c2ccc(O)c(OC)c2)ccc1O]

Curcumin is an active ingredient in the traditional herbal remedies. It is known to have antioxidant and anti-inflammatory properties. It is commonly used for the treatment of infections, burns, acne, wound dressing, sprains, swelling, asthma, allergy diabetes, cough, sinusitis, flu, rheumatism, liver disorders, abdominal pain associated diseases, skin wounds and inflammations. It is also known to have anti-angiogenic, chemo-therapeutic and immunomodulatory effects.

Curcumin has poor solubility in aqueous solvent because of its non-polar nature. The stability of curcumin in aqueous solution is pH dependent. Aqueous solution of curcumin is not stable under neutral to basic pH (7.5 to 12). In acidic conditions, stability of curcumin is higher. However, curcumin does not disintegrate on entering the blood stream and instead binds to plasma protein and remains in blood circulation, resulting in poor binding & absorption.

Thus, use of curcumin as a therapeutic agent has many challenges owing to its poor binding, stability, absorption into the cell level, poor pH and toxicity and hence not found suitable to enhance the bioavailability of curcumin with desirable dose. Therefore, a formulation of curcumin with better solubility and stability in aqueous medium, thereby increasing its absorption, bioavailability and efficiency is a major challenge for researcher/inventor to overcome.

U.S. Pat. No. 9,878,040 discloses a formulation of curcuminoid with essential oil of turmeric, wherein ar-turmerone is the main constituent of essential oil of turmeric. Weight ratio of the curcuminoid and an essential oil of turmeric ranges from about 1:1 to about 99:1. Although the prior art solves the problem of enhancement of bioavailability of curcumin, the problem of leakage in storage and maintenance of pH of the formulation is a challenging problem. The essential oil of turmeric is highly acidic in nature, which makes the formulation acidic and not preferable in oral dosage form. Also, the essential oil of turmeric causes leakage of soft gel formulation. Further, the formulation of the prior art has very poor solubility in water which impacts bioavailability of the prior art formulation.

Indian patent application number 1776/DEL/2008 discloses curcuminoid formulation comprising curcumin or curcuminoids, a lipidic carrier system with an HLB between 3 and 14, and a pH buffer, which forms a self nanoemulsion dilution with water, gastric fluid, or intestinal fluid of globule size of less than 200 nm. It additionally contains a bio enhancer selected from glucoronidation inhibitor or Cytochrome P 450 or P-glycoprotein inhibitor) inhibitor, alkaloids (e.g., piperine, piperine derivatives, piperidine derivatives), glycosides (e.g., quercetin, flavones derivatives), tannins and mixture. However, the formulation of the aforementioned prior art has poor solubility in water and which results in less bioavailability of the formulation. Further, the antimicrobial activity has been compromised due to dilution of the formulation.

Indian patent application number 3646/CHE/2013 discloses curcumin formulation comprising curcumin mixture and water extract. The curcumin mixture comprises curcumin dry crystals, volatile oil, fixed oil whereas water extract comprises soluble proteins, dietary fibers and carbohydrates extracted from turmeric. However, the formulation of the prior art has poor solubility in water and which results in less bioavailability of the formulation.

In view of the above, it is desirable to provide formulations comprising curcuminoid(s) and/or its derivatives having high solubility in water, high bioavailability and stability, non-acidic pH and no antimicrobial activity, leading to better penetration, absorption and binding to substrate molecules. This would help in overcoming the challenges associated with the using curcuminoid(s) as therapeutic agents, including poor binding & absorption into the cell, poor pH, toxicity and hence having better therapeutic effects.

### OBJECTS OF THE INVENTION

The present invention provides at least a solution to the above limitation(s) identified in prior art(s) associated with use of curcumin/curcuminoids in native form. This would help in overcoming the challenges including poor binding & absorption or curcumin into cell, poor pH generation, toxicity.

Thus, the principal object of the present invention is to provide formulations comprising curcuminoids exhibiting enhanced solubility and bioavailability.

### SUMMARY OF THE INVENTION

In an aspect of the present invention, there is provided a formulation comprising: (a) turmeric extract comprising at least 95% curcuminoids; and, (b) at least one of (i) piperine or derivatives thereof; (ii) bees wax; (iii) acrysol K-140; and (iv) turmeric oil.

This summary is not intended to identify essential features of the claimed subject matter nor is it intended for use in determining or limiting the scope of the claimed subject matter.

### DETAILED DESCRIPTION OF THE INVENTION

The following description details the various embodiments of the subject invention. The embodiments of the invention set forth herein below illustrate the invention but are not to be construed as limiting the scope of the invention.

Those skilled in the art will be aware that the invention described herein is subject to variations and modifications other than those specifically described. It is to be understood that the invention described herein includes all such variations and modifications. The invention also includes all such steps, features, formulations and methods referred to or indicated in this specification, individually or collectively, and any and all combinations of any two or more of said steps or features.

The term “acrysol K-140” refers to PEG-40 hydrogenated castor oil.

The present invention provides a formulation comprising: (a) turmeric extract comprising at least 95% curcuminoids; and, (b) at least one of (i) piperine or derivatives thereof; (ii) bees wax; (iii) acrysol K-140; and (iv) turmeric oil.

In an embodiment of the present invention, the said 95% curcuminoids comprises curcumin having weight concentration in the range of 70-80%, desmethoxycurcumin having weight concentration in the range of 15-25%, and bisdesmethoxycurcumin having weight concentration in the range of 2.5-6.5%.

In an embodiment of the present invention, the said 95% curcuminoids weight concentration in said formulation is in the range of 25-45%, piperine weight concentration in said formulation is in the range of 0.4-0.8%, turmeric oil weight concentration in said formulation is in the range of 30-40%, acrysol K-140 weight concentration in said formulation is in the range of 20-40%, and bees wax weight concentration in said formulation is in the range of 1-4%.

In an embodiment of the present invention, the formulation comprises turmeric extract, piperine, turmeric oil, acrysol, and bees wax. The 95% curcuminoids weight concentration in said formulation is in the range of 25-45%, piperine weight concentration in said formulation is in the range of 0.5-0.7%, turmeric oil weight concentration in said formulation is in the range of 32-42%, acrysol K-140 weight concentration in said formulation is in the range of 20-29%, and bees wax weight concentration in said formulation is in the range of 2-3%. In an embodiment, the 95% curcuminoids weight concentration in said formulation is about 35±1%, piperine weight concentration in said formulation is about 0.6±0.1%, turmeric oil weight concentration in said formulation is about 37±1%, acrysol K-140 weight concentration in said formulation is about 24±1%, and bees wax weight concentration in said formulation is about 2±1%. In an embodiment, the 95% curcuminoids weight concentration in said formulation is about 29±1%, piperine weight concentration in said formulation is about 0.6±0.1%, turmeric of weight concentration in said formulation is about 39±1%, acrysol K-140 weight concentration in said formulation is about 26±1%, and bees wax weight concentration in said formulation is about 2±1%. In an embodiment, the 95% curcuminoids weight concentration in said formulation is about 40±1%, piperine weight concentration in said formulation is about 0.55±0.1%, turmeric oil weight concentration in said formulation is about 34±1%, acrysol K-140 weight concentration in said formulation is about 22±1%, and bees wax weight concentration in said formulation is about 2±1%. It is to be understood that the formulation total weight percentage is made up to 100% by addition of one or more excipients/fillers which are generally considered as being pharmacologically inert, and/or do not substantially influence the solubility and/or bioavailability and/or stability of the turmeric extract.

In an embodiment of the present invention, the formulation comprises turmeric extract, and bees wax. The 95% curcuminoids weight concentration in said formulation is in the range of 25-45%, and bees wax weight concentration in said formulation is in the range of 2-3%. In an embodiment, the 95% curcuminoids weight concentration in said formulation is about 35±1%. In an embodiment, the 95% curcuminoids weight concentration in said formulation is about 29±1%. In an embodiment, the 95% curcuminoids weight concentration in said formulation is about 40±1%. In an embodiment, bees wax weight concentration in said formulation is about 2±1%. It is to be understood that the formulation total weight percentage is made up to 100% by addition of one or more excipients/fillers which are generally considered as being pharmacologically inert, and/or do not substantially influence the solubility and/or bioavailability and/or stability of the turmeric extract.

In an embodiment of the present invention, the formulation comprises turmeric extract, piperine, and bees wax. The 95% curcuminoids weight concentration in said formulation is in the range of 25-45%, piperine weight concentration in said formulation is in the range of 0.5-0.7%, and bees wax weight concentration in said formulation is in the range of 2-3%. In an embodiment, the 95% curcuminoids weight concentration in said formulation is about 35±1%. In an embodiment, the 95% curcuminoids weight concentration in said formulation is about 29±1%. In an embodiment, the 95% curcuminoids weight concentration in said formulation is about 40±1%. In an embodiment, piperine weight concentration in said formulation is about 0.6±0.1%. In an embodiment, bees wax weight concentration in said formulation is about 2±1%. It is to be understood that the formulation total weight percentage is made up to 100% by addition of one or more excipients/fillers which are generally considered as being pharmacologically inert, and/or do not substantially influence the solubility and/or bioavailability and/or stability of the turmeric extract.

In an embodiment of the present invention, the formulation comprises turmeric extract, turmeric oil, and bees wax. The 95% curcuminoids weight concentration in said formulation is in the range of 25-45%, turmeric oil weight concentration in said formulation is in the range of 35-40%, and bees wax weight concentration in said formulation is in the range of 2-3%. In an embodiment, the 95% curcuminoids weight concentration in said formulation is about 35±1%. In an embodiment, the 95% curcuminoids weight concentration in said formulation is about 29±1%. In an embodiment, the 95% curcuminoids weight concentration in said formulation is about 40±1%. In an embodiment, the turmeric oil weight concentration in said formulation is about 37±1%. In an embodiment, bees wax weight concentration in said formulation is about 2±1%. It is to be understood that the formulation total weight percentage is made up to 100% by addition of one or more excipients/fillers which are generally considered as being pharmacologically inert, and/or do not substantially influence the solubility and/or bioavailability and/or stability of the turmeric extract.

In an embodiment of the present invention, the formulation comprises turmeric extract, acrysol K-140, and bees wax. The 95% curcuminoids weight concentration in said formulation is in the range of 25-45%, acrysol K-140 weight concentration in said formulation is in the range of 22-26%, and bees wax weight concentration in said formulation is in the range of 2-3%. In an embodiment, the 95% curcuminoids weight concentration in said formulation is about 35±1%. In an embodiment, the 95% curcuminoids weight concentration in said formulation is about 29±1%. In an embodiment, the 95% curcuminoids weight concentration in said formulation is about 40±1%. In an embodiment, the acrysol K-140 weight concentration in said formulation is about 24±1%. In an embodiment, bees wax weight concentration in said formulation is about 2±1%. It is to be understood that the formulation total weight percentage is made up to 100% by addition of one or more excipients/fillers which are generally considered as being pharmacologically inert, and/or do not substantially influence the solubility and/or bioavailability and/or stability of the turmeric extract.

In an embodiment, the formulation of the present invention can be formulated into soft-gel, liquid, nanocrystals, polymer conjugates, liposomes, solid lipid nanoparticles, dendrimers, polymerosomes, and the like, preferably soft-gels.

In an embodiment, the formulation of the present invention exhibits enhanced aqueous solubility (at various concentrations) compared turmeric extract alone.

In an embodiment, the formulation of the present invention exhibits enhanced bio-absorption.

In an embodiment, the formulation of the present invention exhibits improved bioavailability.

In an embodiment, the formulation of the present invention has higher active ingredient loading.

In an embodiment, the formulation of the present invention has increased absorption of curcuminoids in the blood and better dispensability upon oral consumption.

In an embodiment, the formulation of the present invention has faster bio-uptake.

As disclosed herein the term “curcuminoids” is a mixture of curcumin, demethoxycurcumin and bisdemethoxycurcumin. In some embodiments, the term “curcuminoids” includes pure curcumin wherein curcumin is the major component of the curcuminoid mixture. In some embodiments, the term “curcuminoids” includes “95% curcuminoids”, wherein 95% of the crystals having curcuminoid mixture are composed of curcumin, demethoxycurcumin and bisdemethoxycurcumin.

Piperine is an alkaloid with chemical formula (E,E)-5-(3,4-Methylenedioxyphenyl)-2,4-pentadienoylpiperidide 1Piperoylpiperidine, and structure.

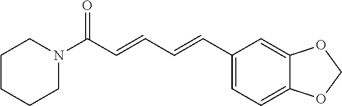 [SMILES: O=C(/C=C/C=C/c1ccc2c(c1)OCO2)N1CCCCC1]

As disclosed herein the term “turmeric oil” refers to “essential oil of turmeric”. It is obtained as a by-product during the extraction of curcumin or curcuminoids from turmeric.

Acrysol K-140 is derived from hydrogenated castor oil and ethylene oxide. The Acrysol K-140 is used as non-ionic oil in water solubilizing and emulsifying agent. It is particularly suitable for fat soluble vitamins and demonstrate exceptional chemical stability. It is almost tasteless marking it ideal for oral application.

The formulation of the present invention may additionally comprise stabilizers, acids/bases for pH adjustment, viscosity modifying agent, salts to adjust the osmolality of the formulation and other pharmaceutically acceptable ingredients.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs.

### Example 1

### Formulations

Formulation 1

| Ingredient | % wt concentration |
|---|---|
| 95% curcuminoids | 35.182 |
| Piperine | 0.627 |
| Turmeric oil | 37.136 |
| Acrysol K-140 | 24.782 |
| Beeswax | 2.271 |

Formulation 2

| Ingredient | % wt concentration |
|---|---|
| 95% curcuminoids | 29.904 |
| Piperine | 0.674 |
| Turmeric oil | 39.97 |
| Acrysol K-140 | 26.973 |
| Beeswax | 2.443 |

Formulation 3

| Ingredient | % wt concentration |
|---|---|
| 95% curcuminoids | 40.46 |
| Piperine | 0.58 |
| Turmeric oil | 34.3 |
| Acrysol K-140 | 22.591 |
| Beeswax | 2.099 |

Formulation 4

| Ingredient | % wt concentration |
|---|---|
| 95% curcuminoids | 35.182 |
| Beeswax | 2.271 |

Formulation 5

| Ingredient | % wt concentration |
|---|---|
| 95% curcuminoids | 35.182 |
| Piperine | 0.627 |
| Beeswax | 2.271 |

Formulation 6

| Ingredient | % wt concentration |
|---|---|
| 95% curcuminoids | 35.182 |
| Turmeric oil | 37.176 |
| Beeswax | 2.271 |

Formulation 7

| Ingredient | % wt concentration |
|---|---|
| 95% curcuminoids | 35.182 |
| Acrysol K-140 | 24.782 |
| Beeswax | 2.271 |

### Example 2

### Preparation of Formulation

a. Acrysol K-140 (^{˜}1 Kg to 40 Kg) was heated at a temperature in the range 50 to 55° C. on a water bath for 15 to 40 minutes;
b. In a separate vessel, turmeric oil was added to piperine powder in the ratio 6:1 to 75:1 with continuous stirring till it dissolved (for about 30 minutes).
c. 95% curcuminoids was added slowly to the solution of step b in the ratio 0.5:1 to 1:1 by weight under continuous stirring up to 2 hours.
d. The contents from step a was then added to the contents of step c under continuous stirring till a viscous solution was obtained, which comprises the formulation of curcuminoids with piperine.

The above formulation may be formulated into various dosage forms like drops, soft gel capsules etc. as provided below.

Please note that the following methods are given as exemplary embodiments only and should not be construed to limit the scope of the present invention.

### Example 2

### Preparation of Gelatin

Gelatin was prepared by the following process:

| Sr. No. | Name of raw material | Qty. per batch (in % w/w) |
|---|---|---|
| 1 | Gelatin | 44% |
| 2 | Glycerin | 10% |
| 3 | Sorbitol 70% | 11% |
| 4 | Annatto Liquid (Natural Color) | 4% |
| 5 | Purified water | 31% |

1. Glycerin, sorbitol, and purified water in the amounts as indicated above was passed through 100# sieve and added to a gelatin melting vessel and mixed for 15 mins.
2. At a temperature of about 70°, gelatin was added to the mixture of step 1.
3. The mixture of step 2 was stirred for about 1 hour under vacuum conditions. Optionally color may be added in this step.
4. The mixture of step 3 was further stirred for about 30 minutes under vacuum to obtain a gelatin mass.
5. The gelatin mass of step 4 was filtered using Filter sieve (40# sieve) to obtain gelatin for preparing capsules comprising formulation of the present invention.

### Example 3

### Preparation of Drops/Gel Capsules

Preparation of formulation as soft gel capsules with formulation (formulation 1) as given in table below:

| Sr. No. | Ingredients | Qty Kg | Qty (% W/W) |
|---|---|---|---|
| 1 | 95% Curcuminoids | 18 | 35.182% |
| 2 | Piperine | 0.321 | 0.627% |
| 3 | Turmeric oil | 19 | 37.136% |
| 4 | Acrysol K-140 | 12.679 | 24.782% |
| 5 | Beeswax | 1.162 | 2.271% |

1. Acrysol K-140 was heated to about 50° C. on a water bath for about 30 minutes;
2. In a separate vessel turmeric oil was added to Piperine powder with continuous stirring till it dissolved (for about 30 minutes);
3. 95% curcuminoids was added slowly to the solution of step 2 under continuous stirring up to 2 hours.
4. The contents of step 1 was then added to the contents of step 3 under continuous stirring till a viscous solution was obtained, which comprised the formulation of curcuminoids with piperine.
5. Melted bees wax of 20 mg per capsule was added to the mixture of step 4 under continuous stirring for 30 minutes;
6. Gelatin was passed to gelatin shell machine where previously prepared formulation was added.
7. From one end, gelatin was passed and from the other, prepared formulation was added. Since the gelatin acquires the shape of oval capsule by having filling of formulation, exactly same capsules of desired quantity were obtained.
8. The product prepared was in the form of yellow capsules with yellow viscous liquid and stable as there was no particle settlement in the formulation and pH of the mixture was found between 6.3 and 6.8.

Preparation of drops formulation of curcuminoids with piperine, with formulation as given below:

| Sr. No. | Ingredients | Qty Kg |
|---|---|---|
| 1 | 95% Curcuminoids | 18 |
| 2 | Piperine | 0.321 |
| 3 | Turmeric oil | 19 |
| 4 | Acrysol K-140 | 12.679 |

1. Acrysol K-140 was heated to about 50° C. on a water bath for about 30 minutes;
2. In a separate vessel, turmeric oil was added to Piperine powder with continuous stirring till it dissolved (for about 30 minutes);
3. 95% curcuminoids was slowly to the solution of step 2 under continuous stirring up to about 2 hours;
4. The contents of step 1 was added to the contents of step 3 under continuous stirring till a viscous solution was obtained, which comprises the formulation as above.

The product was found to be yellow viscous liquid and stable as there was no particle settlement in the formulation and pH was found between 6.3 and 6.8.

### Example 4

### Solubility of Formulations 1-7

Briefly, solutions of formulations 1-7 were prepared by weighing 25 mg of each formulation and added to 50 ml of distilled water, or methanol, or corn oil in conical flask and kept at 37±1° C. in s shaking incubator for about 24 hours. The contents were subsequently filtered through 0.22 μm filter. Different dilutions of filtrate ranging from 5-25 μg/ml were prepared using Krebs ringer phosphate saline with IPA solution (7:3) and absorbance was measured at λmax using 422 nm wavelength.

The percent solubility of Formulations 1-7 compared to standard (95% curcuminoids alone) at various concentrations as above, in methanol (solvent) is provided below.

| Conc. | Formulation | | | | | | | |
|---|---|---|---|---|---|---|---|---|
| (μg/ml) | 1 | 2 | 3 | 4 | 5 | 6 | 7 | Standard |
| 5 | 27.25 | 24.16 | 37.77 | 90.07 | 86.95 | 51.57 | 54.76 | 100 |
| 10 | 30.14 | 25.04 | 36.84 | 89.29 | 89.44 | 51.89 | 53.23 | 100 |
| 15 | 30.79 | 25.57 | 37.17 | 88.55 | 86.64 | 51.41 | 54.56 | 100 |
| 20 | 31.08 | 26.78 | 37.52 | 90.65 | 88.26 | 51.66 | 56.26 | 100 |
| 25 | 34.19 | 29.24 | 40.47 | 93.32 | 92.87 | 54.55 | 58.35 | 100 |

As seen from the table above, it is evident that the various formulations are not effective in enhancing the solubility of 95% curcuminoids in methanol as solvent.

The percent solubility of Formulations 1-7 compared to standard (95% curcuminoids alone) at various concentrations as above, in water is provided below.

| Conc. | Formulation | | | | | | | |
|---|---|---|---|---|---|---|---|---|
| (μg/ml) | 1 | 2 | 3 | 4 | 5 | 6 | 7 | Standard |
| 5 | 60.00 | 120.00 | 240.00 | 300.00 | 300.00 | 400.00 | 280.00 | 100 |
| 10 | 233.33 | 116.66 | 466.66 | 466.66 | 16.66 | 333.33 | 325.00 | 100 |
| 15 | 200.00 | 633.33 | 1066.66 | 766.66 | 333.33 | 433.33 | 833.33 | 100 |
| 20 | 171.42 | 142.85 | 371.42 | 228.57 | 71.42 | 200.00 | 271.42 | 100 |
| 25 | 244.44 | 233.33 | 444.44 | 311.11 | 188.88 | 200.00 | 411.11 | 100 |

As seen from the table above, it can be readily appreciated that the various formulations enhance the aqueous solubility of 95% curcuminoids compared to solubility of 95% curcuminoids alone.

This is particularly important as solubility is an essential factor in ensuring therapeutic applicability of turmeric extract/95% curcuminoids.

The percent solubility of Formulations 1-7 compared to standard (95% curcuminoids alone) at various concentrations as above, in corn oil is provided below.

| Conc. | Formulation | | | | | | | |
|---|---|---|---|---|---|---|---|---|
| (μg/ml) | 1 | 2 | 3 | 4 | 5 | 6 | 7 | Standard |
| 5 | 110.82 | 100.55 | 81.10 | 191.00 | 84.03 | 254.67 | 201.28 | 100 |
| 10 | 90.02 | 81.18 | 63.40 | 126.05 | 123.33 | 186.54 | 193.60 | 100 |
| 15 | 83.13 | 74.01 | 63.28 | 95.23 | 139.69 | 151.72 | 195.17 | 100 |
| 20 | 74.24 | 67.91 | 59.50 | 87.78 | 140.79 | 132.20 | 174.28 | 100 |
| 25 | 60.50 | 59.32 | 62.46 | 72.95 | 127.66 | 114.25 | 134.79 | 100 |

As seen from the table above, certain formulations, particularly formulation 5, 6 and 7 demonstrate enhanced solubility in corn oil compared to 95% curcuminoids alone. This is particularly important as this suggests that the said formulations may be more effective in therapeutic applicability to ensure enhanced uptake of the active ingredient (95% curcuminoids) by cells (cell membranes are comprised mostly of lipids).

### Example 5

### Permeability Study

Comparative ex-vivo kinetic study was carried out using non-everted rat model to ascertain the bio-absorbance of formulation 1.

Briefly, male Wistar rats (weighing 200-250 g) were used. Animals were sacrificed by cervical dislocation after overnight fasting. The small intestine was removed by cutting across the upper end of the duodenum and the lower end of the ileum and manual stripping of the mesentery.

The small intestine was washed out carefully with cold normal oxygenated saline solution (0.9% w/v, NaCl) using a syringe equipped with blunt end. The clean intestinal tract was prepared into 8±0.2 cm long sacs having a diameter of 3±0.5 mm. Each sac [A1-A2 (formulation), C1-C2 (standard)] was filled with 1 ml of formulation or standard via the blunt needle and the two sides of the intestine were tied tightly. Each non-everted intestinal sac was placed in a glass conical flask containing 50 ml of a mixture of Krebs Ringer phosphate saline buffer pH 7.4 and isopropyl alcohol in the ratio of 7:3 (v/v). The entire system was maintained at 37° C. in a shaking water bath operating at about 50 rpm and aerated with oxygen. From outside of the sac, 4 ml samples were withdrawn at various time points and replaced with fresh sample. The samples were analyzed at λmax 422 nm.

% release

| Time (hr) | Formulation 1 | Standard |
|---|---|---|
| 1 | 57.48 | 2.29 |
| 2 | 55.38 | 4.29 |
| 3 | 54.17 | 7.43 |
| 5 | 49.53 | 12.32 |
| 7 | 49.53 | 16.79 |

(Absorbances and mean values are not shown in the table below).

As seen from the table above, it is clearly established that the formulation comprising piperine, turmeric oil, acrosyl and bees wax exhibits significantly higher permeability (cell uptake), thus solving the issue of poor bio-absorption of curcuminoids.

From the table above, it can also be appreciated that the cell uptake by hour 1 of curcuminoids in the form of formulation 1 is about 25-fold more than control (standard). At hour 2, cell uptake curcuminoids in the form of formulation 1 is about 13-fold more than control (standard). At hour 3, cell uptake of curcuminoids in the form of formulation 1 is about 7.3-fold more than control (standard). At hour 5, cell uptake of curcuminoids in the form of formulation 1 is about 4-fold more than control (standard). At hour 7, cell update of curcuminoids in the form of formulation 1 is about 2.9-fold more than control (standard). Overall, it can be seen that curcuminoids uptake in the form of formulation 1 is maximal by 1 hour and seemingly plateaus as seen until hour 7. In contrast, curcuminoids (standard) uptake is very low in hour 1 and only goes up to about 16% by hour 7, which clearly shows that the formulation not only enhances uptake of curcuminoids, but it also reduces the time period of uptake.

It is known the art that drug/active ingredient in an oral dosage typically exhibits better absorption within the span of three hours from the initial intake. In the present instance, it can be seen that the formulation of the present invention exhibits maximal absorption within the first few hours.

### Example 6

### Anti-Microbial Activity

Anti-microbial assay of curcuminoids with piperine was performed using sample of Curcuminoids with piperine soft gel capsule and the analysis result was found as shown below

TABLE 13

| Sr. No. | Test Parameter | Result | Limit/Specification |
|---|---|---|---|
| 1 | E. coli | Absent | Absent/10 gm |
| 2 | Samonella | Absent | Absent/10 gm |
| 3 | S. Aureus | Absent | Absent/10 gm |
| 4 | p. Aeruginosa | Absent | Absent/10 gm |
| 5 | C. Albicans | Absent | Absent/10 gm |
| 6 | Bile Tolerant gram negative | Absent | Absent/10 gm |
| | bacteria | | |

The above table describes that the anti-microbial activity performed on the soft gel capsules, analysis shows that there's absence of all given bacteria's which makes the formulation best suited for oral absorption.

### Advantages of the Invention

The formulation of the present invention relates to formulation comprising 95% curcuminoids.

The various formulations (formulations 1-7) enhance the solubility, and bio-availability of curcumin. Acrysol K-140 (PEG 40 hydrogenated castor oil) also addresses the problem of leakage and maintains pH of the formulation between 6.3 and 6.8. The formulation is useful as an anti-inflammatory and anti-oxidant in treatment of various diseases. Further the formulation of curcuminoids not only increases the bioavailability of curcumin, but also solves the problem of leakage in the formulation if filled in softgel as well as it maintains the pH of the formulation which is required to be neutral. The formulations comprising curcuminoids with piperine possess high solubility in water, higher bio-absorption, improved bioavailability and higher drug loading ability as compared to native formulation of curcumin and curcuminoid formulations of the prior art. The formulation of the present invention shows better solubility, stability, penetration and better efficacy which shall be advantageous for various health related purposes such as medicine, dietary, food or cosmetic formulations.

We claim:

1. A formulation comprising:
a. turmeric extract comprising at least 95% curcuminoids; and
b. at least one of:
i. piperine or derivatives thereof;
ii. bees wax;
iii. acrysol K-140; and
iv. turmeric oil.

2. The formulation as claimed in claim 1, wherein said 95% curcuminoids comprises curcumin having weight concentration in the range of 70-80%, desmethoxycurcumin having weight concentration in the range of 15-25%, and bisdesmethoxycurcumin having weight concentration in the range of 2.5-6.5%.

3. The formulation as claimed in 1, wherein 95% curcuminoids weight concentration in said formulation is in the range of 25-45%, piperine weight concentration in said formulation is in the range of 0.4-0.8%, turmeric oil weight concentration in said formulation is in the range of 30-40%, acrysol K-140 weight concentration in said formulation is in the range of 20-40%, and bees wax weight concentration in said formulation is in the range of 1-4%.

4. The formulation as claimed in claim 1, comprising turmeric extract, piperine, turmeric oil, acrosyl, and bees wax, wherein 95% curcuminoids weight concentration in said formulation is in the range of 25-45%, piperine weight concentration in said formulation is in the range of 0.5-0.7%, turmeric oil weight concentration in said formulation is in the range of 32-42%, acrysol K-140 weight concentration in said formulation is in the range of 20-29%, and bees wax weight concentration in said formulation is in the range of 2-3%.

5. The formulation as claimed in claim 1, comprising turmeric extract, and bees wax, wherein 95% curcuminoids weight concentration in said formulation is in the range of 25-45%, and bees wax weight concentration in said formulation is in the range of 2-3%.

6. The formulation as claimed in claim 1, comprising turmeric extract, piperine, and bees wax, wherein 95% curcuminoid weight concentration in said formulation is in the range of 25-45%, piperine weight concentration in said formulation is in the range of 0.5-0.7%, and bees wax weight concentration in said formulation is in the range of 2-3%.

7. The formulation as claimed in claim 1, comprising turmeric extract, turmeric oil, and bees wax, wherein 95% curcuminoids weight concentration in said formulation is in the range of 25-45%, turmeric oil weight concentration in said formulation is in the range of 35-40%, and bees wax weight concentration in said formulation is in the range of 2-3%.

8. The formulation as claimed in claim 1, comprising turmeric extract, acrysol K-140, and bees wax, wherein 95% curcuminoids weight concentration in said formulation is in the range of 25-45%, acrysol K-140 weight concentration in said formulation is in the range of 22-26%, and bees wax weight concentration in said formulation is in the range of 2-3%.

9. The formulation as claimed in claim 1, optionally further comprising suitable excipients.

10. The formulation as claimed in claim 1, wherein said formulation is suitable as an oral supplement, preferably in the form of a gel capsule.

11. A preparation of turmeric extract with enhanced aqueous solubility, said preparation essentially comprising a formulation as claimed in claim 1.

12. A preparation of turmeric extract with enhanced permeability, said preparation essentially comprising a formulation as claimed in claim 1.
